(19) United States
(12) Patent Application Publication
(10) Pub. No.: US 2007/0260101 A1
(43) Pub. Date: Nov. 8, 2007

(54) Membrane reactor process for chemical conversions

(75) Inventors: Martin E. Carrera, Naperville, IL (US); Luis A. Baez Linde, Holden, MA (US); Mark S. Kleefisch, Plainfield, IL (US); Jane Zhang, Naperville, IL (US)

Correspondence Address: INEOS USA LLC, 3030 WARRENVILLE RD, S/650, LISLE, IL, 60532, US

(73) Assignee: Innovene USA LLC

(21) Appl. No.: 11/415,566

(22) Filed: May 2, 2006

(51) Int. Cl. C07C 5/327; C07C 5/373

(52) U.S. Cl. 585/654; 585/658

(57) ABSTRACT

Processes for chemical conversion of volatile organic compounds to value added products using multiphasic membrane reactors are described. More particularly the multiphasic materials comprising two or more phases bound to one another which in the form of a solid state membrane demonstrate an ability to selectively convey electrons, hydrogen and oxygen between different gaseous mixtures. Characteristics of such multiphasic materials provide independent, controllable, counter-current transport of hydrogen, electrons and oxygen.

### TECHNICAL FIELD

The present invention relates to processes for chemical conversion of volatile organic compounds to value added products using membrane reactors, and more particularly to processes using dense membranes of multiphasic materials that provide independent, controllable, counter-current transport of hydrogen, electrons and oxygen. The invention includes processes for dehydrogenation of organic compounds, such as hydrocarbons and alcohols. Processes of the invention are advantageously used with catalysts, for example dehydrogenation catalysts for converting alkanes to corresponding alkene products. Catalysts used to facilitate the chemical conversion reactions are generally either on the membrane as a layer or in reaction zone as a bulk material.

### BACKGROUND OF THE INVENTION

Alkenes, commonly known as olefins, are used to produce many useful polymers and as components of numerous synthetic chemicals. Ethylene is used in one of several forms of polyethylene, as ethylene glycol to make polyester, in the manufacture of vinyl acetate and vinyl chloride, as a building block for linear alpha olefins, and in the production of styrene. Propylene is used in the synthesis of polypropylene, propylene oxide, acrylonitrile, and cumene. Butadiene is used primarily to make elastomers including styrene-butadiene rubber, neoprene, and nitrile rubber. The olefins/polymers value chain is typically composed of several distinct steps: (1) conversion of hydrocarbons including alkanes into alkenes, (2) in some cases transformation of the alkenes into intermediate products via oxidation, ammoxidation, or alkylation (e.g. acrylonitrile, styrene, and cumene), (3) polymerization or oligomerization into macromolecules, and (4) final device fabrication into end products.

Several commercialized methods are practiced to synthesize olefins. The most industrially significant method is steam cracking. Steam crackers can produce olefins from numerous hydrocarbon feeds including natural gas liquids, light petroleum gases, light paraffinic naphthas, and mixtures thereof. Commercialized steam cracking processes utilize high temperature pyrolysis where these feeds are mixed with steam and heated to temperatures in a range from about 700 to 900° C. Thermodynamic equilibrium limits olefin yield to relatively low amounts. The olefins industry has gotten above this constraint by pushing temperatures up to where free radical mechanisms start to occur. The industry relies on high temperatures and quick contact time so that free radical reactions start and then are quickly quenching the reactants to focus the yield pattern on olefins and limit the formation of byproducts. Reactor development in conventional olefins crackers has been oriented toward shorter and shorter contact times with large quench heat exchangers to quickly stop the reactions. More detail regarding the operation, engineering, and optimization of steam cracking may be found in Ullmann's Encyclopedia of Industrial Chemistry.

When an olefin is made from an alkane, commonly known as paraffin, hydrogen is also produced. Thermodynamics dictates the maximum yield of olefins and hydrogen possible at a specific reactor temperature. Chemical conversions approach but do not exceed the thermodynamic equilibrium limit. See for example, U.S. Pat. No. 6,271,431, in the name of Christian Busson, Jean-Pierre Burzynski, Henri Delhomme, and Luc Nougier, describes a reactor that produces ethylene yields higher than those normally obtained in commercial cracking reactors by lowering the temperature and increasing the contact time of the process. Their process approaches but cannot exceed the thermodynamic equilibrium limit.

U.S. Pat. No. 6,111,156, in the name of Michael C. Oballa, David Purvis, Andrzej Z. Krzywicki, and Leslie W. Benum, describes a high temperature, high conversion olefin process that approaches the maximum thermodynamic yield of olefins. The patent describes furnace tubes or coils that have been adapted to operate at temperatures higher than those typically employed in conventional steam crackers (above 1050° C.), thereby increasing conversion. Examples of these adaptations include coatings available from Surface Engineered Products and ceramic tubes including silicon carbide.

There are several problems with this approach to increasing olefin yield. Joining silicon carbide to metals is very difficult and the technology for doing so, and keeping the joint in tact at these temperatures (above 1050° C.), is not well developed. Therefore, this leads to frequent ceramic tube failures and generally unreliable operations. Furthermore, vibrations typically encountered during steam cracking operations can easily damage and destroy silicon carbide tubes at the elevated temperatures described in U.S. Pat. No. 6,111,156. Olefin selectivity is believed to be poor at these elevated temperatures. If the operation of the steam-cracking reactor is too severe, numerous researchers have pointed out that the amount of olefin produced per pound of feed converted can actually level out and even decrease. For example, a kinetic severity factor (KSF) is defined in “Pyrolysis: Theory and Industrial Practice” edited by L. F. Albright and coworkers and published by Academic Press in 1983 that relates reactor residence time, reactor temperature, reactor pressure, quenching, and feedstock type. They show that the concentration of olefins passes through a maximum as KSF is increased. This occurs because secondary reactions that begin to consume olefins play a larger role at high severities. The amount of undesirable byproducts is also understood to be high when steam cracking at the elevated temperatures described in U.S. Pat. No. 6,111,156.

Alternative routes for the production of ethylene, propylene and butylenes have been of interest for many years as an alternative to steam cracking. Note that all of these can approach but not exceed the thermodynamic conversion limit. The most feasible route to the commercial scale on-purpose production of these alkenes has generally been through the catalytic dehydrogenation of the relevant alkane according to the formula
CₙH₍₂ₙ₊₂₎---------->CₙH₍₂ₙ₎+H₂
where n is an integer greater than or equal to 2. Catalytic dehydrogenation reactions are limited by thermodynamic constraints resulting from the highly endothermic nature of the reaction. As reactor temperatures increase above 600° C., side-cracking reactions based on free radical mechanisms can occur, leading to the formation of lighter hydrocarbons and coke. Employing a catalyst reduces the required reaction temperature and thereby largely avoids the formation of free radical species in the reactor. The high costs of the alkane feedstock (e.g., ethane, propane, etc.) and the capital required for the dehydrogenation processes make it economically desirable to achieve the highest possible selectivity to alkanes and to limit the formation of coke and coke precursors within the dehydrogenation reactor.

As in catalytic reforming, coke formation on and the resulting deactivation of the dehydrogenation catalyst is reduced by the addition of small amounts of dihydrogen to the dehydrogenation reactor feed. Significant research has been devoted to minimizing the coke formation reaction and in studying the kinetics of coke formation. For instance, R. L. Mieville (Studies in Surface Science and Catalysis, vol. 68, Catalyst Deactivation 1991, pp. 151-159) has showed that the rate of coke formation for a Pt/Al₂O₃ catalyst used in reforming obeys the following equation
rcoke=(A)*(1/pH2)*(pfeed^{0.75})*(1/coke)*(exp(−37000/RT))
Where pH₂ is the partial pressure of hydrogen, pfeed is the partial pressure of the hydrocarbon feed, and “coke” relates to the amount of coke already present on the catalyst. This equation shows that the rate of coke formation is inversely proportional to the hydrogen partial pressure. Without the addition of hydrogen, most dehydrogenation catalysts deactivate in a time frame that is not commercially viable. Typically, in catalytic dehydrogenation processes, the amount of hydrogen added with the reactant alkane for coke suppression is balanced against the reduction in equilibrium conversion brought about by the resulting higher hydrogen concentration. Even with hydrogen addition to the reactor feed, some coke is formed on the catalyst and all commercial catalytic dehydrogenation technologies employ a reactor configuration that is designed to include periodic regeneration of the catalyst.

The extent of the conversion of hydrocarbons to olefins in conventional dehydrogenation systems is typically limited by thermodynamic equilibrium. There is a need for processes that overcome this thermodynamic limit. Removal of this thermodynamic limitation would allow higher per-pass conversion of the hydrocarbon to take place, resulting in a more efficient overall process.

One method that can be employed to remove the thermodynamic limitation is to employ oxidative dehydrogenation of the alkane. Oxidative dehydrogenation of ethane to ethylene has been reviewed recently by Dai et al. (Current Topics in Catalysis, 3, 33-80 (2002)). In an oxidative dehydrogenation process oxygen is added to the dehydrogenation reactor feed and reacts with the hydrogen produced during the dehydrogenation reaction. The hydrogen is converted to water, thereby removing it from the reaction zone and driving the thermodynamic equilibrium to higher alkane conversion values. The heat provided by the exothermic oxidation of hydrogen also can balance the heat required by the endothermic dehydrogenation reaction.

While the concept of oxidative dehydrogenation is not new, to date the process has not been commercialized for the large-scale production of light olefins. There are a number of drawbacks to the use of oxidative dehydrogenation as compared to standard catalytic dehydrogenation. First, addition of oxygen to the feed typically leads to a reduced selectivity to the desired olefin product. Formation of carbon oxides and oxygenated compounds through the undesirable partial combustion of the hydrocarbon feed can lead to lower feed utilization and more complex downstream separation requirements for oxidative dehydrogenation processes. Second, mixing of oxygen with the hydrocarbon feed presents a safety concern that is not present in conventional catalytic dehydrogenation processes. While these risks can be mitigated through the application of safe engineering and design principles and additional safety systems, these systems and procedures can increase the cost and complexity of the oxidative dehydrogenation process and in any case the risks cannot be entirely removed. Finally, presence of both exothermic oxidation and endothermic dehydrogenation reactions within the reactor presents a significant reactor design challenge with regard to the management of heat within the reactor.

It is believed that the most promising way at present to remove the thermodynamic limitation of olefin production is to employ membranes capable of removing hydrogen. Removal of hydrogen causes the chemical reaction to proceed to the right through the law of mass action, thereby achieving much higher conversions, up to 100 percent conversion.

Membranes have been explored that remove hydrogen and thereby allow higher yields of olefins to be achieved. For example, U.S. Pat. No. 3,290,406 describes the use of palladium alloy tubes to remove hydrogen formed during the dehydrogenation of ethane. Membranes made out of palladium or palladium alloys are the most widely explored membranes for hydrogen separations. There are numerous reports in the art of palladium or palladium alloy membranes demonstrating high hydrogen permeation rates and hydrogen selectivities.

However, issues remain to be solved before palladium or palladium alloy membranes can be used in an industrial setting, as pointed out in an article by Collins and coworkers entitled “Catalytic Dehydrogenation of Propane in Hydrogen Permselective Reactors” in Industrial Engineering and Chemistry Research, volume 35, pages 4398-4405 (1996). Collins and coworkers found that palladium membranes deactivated rapidly when placed in alkane dehydrogenation service. Their membranes failed because of a large deposition of coke on the surface of the palladium membrane.

U.S. Pat. No. 5,202,517, in the name of Ronald G. Minet, Theodore T. Tsotsis and Althea M. Champagnie, appears to describe a way to overcome the coking problems associated with palladium membranes by use of porous ceramic membranes impregnated on the surface with palladium or platinum which are contacted with a mixture of alkane and hydrogen. They state that the hydrogen in the feed is needed to suppress the formation of coke.

Another way to suppress the formation of coke on the surface of a hydrogen membrane reactor is to supply a source of oxygen to the membrane reactor in the form of pure dioxygen (diatomic oxygen), air, or steam. However, supplying diatomic oxygen or air to the feed side of a hydrogen membrane reactor would suffer from the same drawbacks associated with oxidative dehydrogenation, namely safety concerns and reduced selectivity to the desired olefin product through the formation of carbon oxides.

It is therefore a general object of the present invention to provide an improved process which overcomes the aforesaid problem of prior art methods for chemical conversion of volatile organic compounds to value added products using membrane reactors.

An improved method for conversion of alkanes to corresponding alkenes should provide better ways to introduce oxygen into the reactor in order to keep the membrane free of coke.

There is a need for membranes that are capable of simultaneously transporting hydrogen and oxygen. For the synthesis of alkenes from alkanes, it is important to carefully balance the rates of oxygen and hydrogen transport. High hydrogen transport is desirable to maximize the production of olefins. Oxygen transport needs to be sufficient to prevent coking problems but not so high as to oxidize the alkane feedstock and form carbon oxides.

Membrane compositions have been described for transport of electrons and hydrogen. Other membrane compositions have been described for conducting electrons and oxygen. Membranes composed of a single phase capable of simultaneous hydrogen and oxygen transport have been described. For example, membranes composed of a single mixed oxide for oxygen and hydrogen transport are described in an article entitled “Oxide Ion Conduction in Ytterbium-Doped Strontium Create” by N. Bonanos, B. Ellis and M. N. Mahmood in Solid State Ionics, vol. 28-30, pages 579-579 (1988). A single phase mixed membrane for alkane dehydrogenation is described in U.S. Pat. No. 5,821,185, U.S. Pat. No. 6,037,514 and U.S. Pat. No. 6,281,403 each in the name of in the name of James H. White, Michael Schwartz and Anthony F. Sammels and assigned to Eltron Research, Inc.

U.S. Pat. No. 6,332,964 in the name of Chieh Cheng Chen, Ravi Prasad, Terry J. Mazanec and Charles J. Besecker describes membranes composed of an electron conducting phase and an oxygen-conducting phase. There are distinct advantages associated with employing such a matrix as a membrane in a reactor. A known problem in a reactor of this type is the slow buildup of coke on the alkane side of the reactor. Using a membrane matrix that conducts oxygen ions may reduce, or even eliminate, coking problems. It is believed that oxygen can be transported from the air side of the membrane to the alkane side where it may reacts with coke precursors as they are formed on the membrane surface. Reaction of the coke precursors with oxygen also provides heat to fuel the endothermic dehydrogenation reaction. Another use for the oxygen that is transported through such a matrix is to react with hydrogen to produce heat, as is needed in steam reforming.

U.S. Pat. No. 6,066,307 in the name of Nitin Ramesh Keskar, Ravi Prasad and Christian Friedrich Gottzmann describes a process for preparing synthesis gas and hydrogen gas using a membrane reactor having two membranes, one membrane that is an oxygen conductor and the other membrane that is a proton conductor, to produce hydrogen gas and synthesis gas.

Dual phase membranes offer the potential to balance the rates of oxygen and hydrogen transport. If one phase is responsible for hydrogen transport and the other is responsible for oxygen transport, it would be possible to adjust the relative amounts of the two phases to maximize hydrogen transport while maintaining an oxygen transport rate sufficient to keep the membrane from coking but not so high as to oxidize the alkane feedstock. It is harder to achieve this balance in single-phase membranes. There is a need for dual phase membranes that conduct both hydrogen and oxygen in order to produce a membrane reactor that facilitates chemical conversions without deactivating too rapidly. For example, improvements in steam reforming and alkane dehydrogenation would be expected if these dual phase membranes were employed.

Alkene production technologies described above produce, along with the desired olefin products, a variety of unwanted or lower-value co-products. For example, in the cracking of hydrocarbons to produce ethylene and propylene, co-products such as methane, hydrogen, acetylene, and others are produced. Likewise, dehydrogenation of alkanes to produce the corresponding olefin also produces co-products such as hydrogen, diolefins and acetylenics. Such co-products make necessary one or more separation and purification steps so that a purified olefin product, suitable for downstream processing, can be obtained.

There is extremely wide scope for the design of such separation systems, limited only by the purity specifications of the final olefins product, technical feasibility, and economic viability. In a practical sense, however, such separation systems have nearly all contained at least the steps of compression of the olefin-containing reactor effluent stream, chilling and partial condensation of the compressed stream, and vapor/liquid separation wherein the liquid contains the olefin product and the vapor contains less valuable lighter gases.

For example and with respect to separation of products from the dehydrogenation of alkanes, U.S. Pat. No. 6,333,445 in the name of John V. O'Brien described a cryogenic separation system for the recovery of olefins from a dehydrogenation process. This process included compression of the dehydrogenation reactor effluent and multiple levels of chilling and subsequent vapor/liquid separation. U.S. Pat. No. 5,026,952 in the name of Heinz Bauer describes a process for recovering C₂+, C₃+ or C₄ hydrocarbons from a high-pressure stream containing these components and lighter components. This process included multiple chilling and vapor/liquid separation stages, as well as rectification and expansion steps. U.S. Pat. No. 5,177,293 in the name of Michael J. Mitariten and Norman H. Scott describes a process for the separation and recovery of product streams from a dehydrogenation reactor that uses a pressure swing adsorption process to concentrate the olefin product. This process also comprises the steps of compression of the dehydrogenation reactor effluent, chilling of the compressed effluent, and subsequent vapor/liquid separation.

With respect to the separation of products from a hydrocarbon cracking reactor, a variety of commercial processes are offered by various technology vendors, including ABB Lummus Global, Kellogg Brown & Root, Inc., Linde A. G., Stone and Webster, Inc., and Technip-Coflexip, among others. A summary of the commercially available steam cracking and product purification technologies from these vendors has been published recently (Hydrocarbon Processing, March 2003, pp. 96-98). While there are many and significant differences in the ethylene production and recovery processes offered by these vendors, it is clear to those skilled in the art that each of the ethylene production and purification processes contains at least the steps of compression of the furnace effluent, and the subsequent chilling and partial condensation of the compressed effluent to produce at least one olefin-rich liquid stream.

It is clear from the discussion above that the majority of separation processes which are necessary in the production of a purified olefin product from a reactor effluent comprise the steps of a) compression of the majority of the reactor effluent, b) chilling and partial condensation of the majority of the compressed reactor effluent stream, and subsequent separation of the resulting liquid and vapor phases to produce an olefin-rich liquid. These steps are common to essentially all light olefin-producing processes primarily because in the cases of dehydrogenation and steam cracking the reactor effluent exits the reactor at relatively low pressure, and because the subsequent purification of the desired olefin product is carried out largely through distillation and vapor/liquid separation. It would therefore be highly desirable to provide a reactor effluent stream which allows the subsequent steps of compression, chilling with partial condensation, and vapor/liquid separation to be carried out in a more energy- and capital-efficient manner.

Commercialized steam cracking processes utilize high temperature pyrolysis where these feeds are mixed with steam and heated to 700° C. to 900° C. During the process, less than 100 percent of the feed is converted per pass because of thermodynamic limitations and in order to maximize the yield of the desired olefin product. Complex and expensive separation equipment is used to recover the unreacted feed from the olefin products and byproducts. The unconverted feed is recycled where it is often remixed with fresh feed. This recycle introduces inefficiency to the olefin process. It would be desirable to reduce this inefficiency by increasing per-pass conversion and thereby reducing or even eliminating recycles. Benefits of higher conversion olefins processes include reducing the size of equipment and capital employed in olefins manufacture, lowering the energy required to produce the olefin product, and elimination of large pieces of equipment devoted to separating the olefin product from unreacted alkane.

Other objects and advantages of the invention will become apparent upon reading the following detailed description and appended claims.

### SUMMARY OF THE INVENTION

Economical processes are disclosed for chemical conversion of volatile organic compounds to value added products using dense membranes of multiphasic materials that provide independent, controllable, counter-current transport of hydrogen, electrons and oxygen.

In one aspect the invention is a process for chemical conversion of volatile organic compounds to value added products, which process comprises: (a) providing a solid state membrane having first surface and opposite thereto a second surface, the membrane comprising a multiphasic material that demonstrates an ability to selectively convey electrons, hydrogen and oxygen between different gaseous mixtures; (b) contacting the first surface of the membrane with gaseous organic stream, substantially free of dioxygen, comprising one or more volatile hydrocarbon; (c) contacting the second surface of the membrane with a gaseous non-organic composition, substantially free of dihydrogen and organic compounds, comprising at least 5 percent by volume of dioxygen; (d) converting, at elevated temperatures, one or more volatile compound in the organic stream to products of conversion comprising corresponding value added organic products, carbonaceous co-products, and hydrogen; (e) permitting at least a portion of the hydrogen co-product to be selectively conveyed through the membrane, from the first surface to the second surface, and oxidizing the conveyed hydrogen; and (f) permitting a predetermined amount of oxygen, in the form of oxygen ions, to be selectively conveyed through the membrane from the second surface to the first surface.

At least a portion of the carbonaceous co-products are oxidized, on or near the first surface, by oxygen selectively conveyed through the membrane from the second surface.

The conversions of the invention advantageously are carried out in the presence of a hydrocarbon conversion catalyst at elevated temperatures in a range from about 400° C. to about 900° C.

The flux of the co-product hydrogen conveyed through the membrane, from the first surface to the second surface of membrane beneficially is at least 1 cm³/min. at standard conditions per cm² of membrane area. Generally according to the invention, the flux of oxygen conveyed through the membrane from the second surface to the first surface is no more than the counter-current flux of hydrogen. Advantageously, the flux of oxygen conveyed through the membrane from the second surface to the first surface is no more than about one-fifth of the counter-current hydrogen flux, and even less than one-tenth for best results.

For hydrocarbon dehydrogenation and oligomerization reactions, suitable catalysts include; oxides of the first row transition metals of a support, for example on an alkali metal oxide; one or more metal selected from the group consisting of nickel, iron platinum, silver and palladium; and perovskite compounds represented by the formula
(D_{1-y}M_{y})EO_{δ}
where D is a metal selected from the group consisting of cerium, terbium, praseodymium and thorium; M comprises at least one other metal selected from the group consisting of titanium, vanadium, chromium, manganese, iron, cobalt, copper and nickel; and E is at least one element selected from the group consisting of magnesium, calcium, strontium, and barium; y is a number such that
0.02<y≦0.5
and δ is a number that renders the compound charge neutral.

In yet another aspect of the invention is a hydrocarbon dehydrogenation process for the production of olefins, the process comprising: (a) providing a gaseous feedstream consisting predominantly of volatile alkane compounds substantially free of dihydrogen and/or dioxygen; (b) providing a solid state membrane having first surface and opposite thereto a second surface, the membrane comprising a non-homogenous, multiphasic solid containing a first phase comprising a metal, alloy or mixed-metal oxide, and a second phase comprising a mixed metal oxide ceramic, wherein the first and second phases are bound to one another and distributed, in a physically distinguishable form, throughout the continuous, fine-grained, second phase, wherein at least one of the bound phases demonstrates an ability to selectively convey hydrogen, another phase demonstrates an ability to selectively convey oxygen ions between different gaseous mixtures, and one or more of the phases demonstrates electronic conductivity; (c) converting, under conditions of reaction for hydrocarbon dehydrogenation at elevated temperatures in a reaction mixture, one or more alkane hydrocarbon in the gaseous feedstream to products of dehydrogenation comprising corresponding value added alkene hydrocarbons, carbonaceous co-products, and hydrogen; (d) contacting the first surface of the membrane with gaseous reaction mixture; (e) contacting the second surface of the membrane with a gaseous non-organic composition, substantially free of dihydrogen and organic compounds, comprising from about 5 upward to about 30 percent by volume of dioxygen; (f) permitting a predetermined amount of oxygen, in the form of oxygen ions, to be selectively conveyed through the membrane from the second surface to the first surface; and (g) permitting at least another portion of the co-product hydrogen to be selectively conveyed through the membrane, from the first surface to the second surface, and oxidizing the conveyed hydrogen.

Gaseous feedstreams of the present invention include utilizing alkanes containing from 2 to 8 carbon atoms, naphtha, raffinate, atmospheric gas oil, vacuum gas oil, distillate, crude oil, crude resids, and mixtures thereof. The desired alkene products according to the present invention comprise ethylene, propylene, one or more isomers of butene, or a combination thereof, and are dependent on the alkane feed type.

In one aspect of the invention, the gaseous feedstream comprises volatile alkane compounds having from about 1 to about 8 carbon atoms, and the conversions are carried out at elevated temperatures in a range from about 400° C. to about 900° C. and pressures in a range upward from about 15 psia to about 500 psia. Desired value added product include alkene compounds comprise at least one member of the group consisting of ethylene, propylene, and isomers of butene. Processes of the invention are advantageously used with catalysts, for example dehydrogenation catalysts for converting alkanes to corresponding alkene products. Catalysts used to facilitate the chemical conversion reactions are generally either on the membrane as a layer or in reaction zone as a bulk material.

In processes of the invention, the gaseous non-organic composition can further comprise at least one member of the group consisting of dinitrogen and carbon dioxide.

Yet another aspect of the invention is a dehydrogenation process for the production of olefin hydrocarbons from a gaseous feedstream consisting predominantly of volatile alkane compounds, the process comprising: (a) providing apparatus comprising a plurality of membrane modules each including first and second zones separated by a membrane comprising a multiphasic material comprising a non-homogenous, multiphasic solid containing a first phase comprising a metal, alloy or mixed-metal oxide, and a second phase comprising a mixed metal oxide ceramic, wherein the first and second phases are bound to one another and distributed, in a physically distinguishable form, throughout the continuous, fine-grained, second phase, wherein at least one of the bound phases demonstrates an ability to selectively convey hydrogen, another phase demonstrates an ability to selectively convey oxygen ions between different gaseous mixtures, and one or more of the phases demonstrates electronic conductivity, each first zone having at least one inlet and outlet for flow of fluid in contact with the membrane, and contiguous with the opposite side thereof a second zone having at least one outlet for flow of another fluid; (b) introducing a feedstream comprising volatile alkane compounds, substantially free of dihydrogen and/or dioxygen into the first zone of one or more of the modules; (c) introducing a gaseous non-organic stream substantially free of dihydrogen and organic compounds, and comprising from about 5 upward to about 30 percent by volume of dioxygen into the second zone of one or more of the modules; (d) converting, under conditions of dehydrogenation at elevated temperatures in the first zone of one or more of the modules, one or more alkane hydrocarbon in the feedstream to corresponding value added alkene hydrocarbons, carbonaceous co-products, and hydrogen; (e) permitting at least a portion of the hydrogen co-product to be selectively conveyed through the membranes, from the first zones into the second zones; and (f) permitting a predetermined amount of oxygen, in the form of oxygen ions, to be selectively conveyed through the membrane from the second zones into the first zones. In processes of the invention, conversions of alkane hydrocarbon in the feedstream carried out in the first zones are beneficially at least 75 molar percent.

In processes of the invention, the first phase can comprise a metal selected from the group consisting of silver, palladium, platinum, gold, rhodium, titanium, nickel, ruthenium, tungsten, and tantalum. In another aspect of the invention, the first phase comprises a ceramic selected from the group consisting of a praseodymium-indium oxide mixture, niobium-titanium oxide mixture, titanium oxide, nickel oxide, tungsten oxide, tantalum oxide, ceria, zirconia, magnesia, or a mixture thereof.

In another aspect of the invention, the second phase comprises a mixed conducting oxide composition represented by
(A_{1-y}A′_{y})(B₁₋ₓB′_{z}B″_{x-z})O_{δ}
where A is a lanthanide element, yttrium (Y), or mixture thereof, A′ is one or more alkaline earth metal; B is iron (Fe); B′ is chromium (Cr), titanium (Ti), or mixture thereof and B″ is manganese (Mn), cobalt (Co), vanadium (V), nickel (Ni), copper (Cu) or mixture thereof; and x and y are each independently selected numbers from zero to about one, and z is a number zero to x; and 6 is a number determined from stoichiometry that renders the compound charge neutral.

In another aspect of the invention, the second phase comprises a mixed cerium oxide composition represented by
M′CeO_{δ}
where M′ is selected from the group consisting of yttrium (Y) and elements having atomic numbers from 58 to 71 inclusive, and δ is a positive number determined from stoichiometry.

In still another aspect of the invention, the second phase comprises a mixed zirconium oxide composition represented by
MƒZrO_{δ}
where M″ is selected from the group consisting of calcium (Ca), yttrium (Y) and elements having atomic numbers from 58 to 71 inclusive, and δ is a positive number determined from stoichiometry.

A flux of the co-product hydrogen conveyed through the membrane, from the first zones to the second zones of the modules is beneficially at least 1 cm³/cm²/min, and a flux of oxygen conveyed through the membrane from the second zones to the first zones is no more than about one-tenth of the counter-current hydrogen flux.

Processes of the invention can comprise at least a plurality of the membrane modules that further comprise structures that during operation facilitate an external path for flow of electrons.

The invention includes processes for dehydrogenation of organic compounds; for example, alkane hydrocarbons to form one or more desired olefin, such as are typically produced by thermal cracking of suitable hydrocarbon feedstock.

For a more complete understanding of the present invention, reference should now be made to the embodiments illustrated in greater detail in the accompanying drawing and described below by way of examples of the invention.

### BRIEF DESCRIPTION OF THE INVENTION

Processes of this invention are suitable for chemical conversion of volatile organic compounds to value added products at high conversion and high selectivity by use of dense membranes comprising multiphasic materials that provide independent, controllable, counter-current transport of hydrogen, electrons and oxygen. Generally a multiphasic solid state membrane that transports oxygen, hydrogen, and electrons, separates a chamber, where alkane dehydrogenation occurs to produce alkenes and hydrogen, from another chamber containing oxygen rich gas. By means of a selective transport of hydrogen out of the dehydrogenation zone through the membrane, and hydrogen combustion in the chamber by oxygen transported in through the membrane, the established chemical equilibrium is shifted, resulting in the production of more hydrogen and by consequence, more alkenes.

In embodiments of the invention where hydrogen flux control is required, structures that facilitate an external path for flow of electrons can be used to increase or decrease hydrogen flux through voltage control.

Processes of the invention allow higher total conversion, at lower temperatures, than state of the art steam pyrolysis reactors, because reactor residence time is not controlled by very fast free radical kinetics. Lower temperatures provide higher selectivity to desired alkene products. Therefore, overall yield is also higher than state of the art pyrolysis reactors. This process is a step change from the traditional selectivity-conversion operating curves and approaches the theoretical limits of such a curve. Combustion of hydrogen in the reactor provides at least a part of the endothermic heat of reaction.

Transport membranes useful in flow reactors of the invention comprise a sintered homogenous mixture of a ceramic composition and a metal that demonstrates an ability to selectively convey hydrogen. Useful metals include palladium, niobium, tantalum, vanadium, or zirconium or a binary mixture of palladium with another metal such as niobium, silver, tantalum, vanadium, or zirconium. Membrane materials advantageously comprise at least one mixed metal oxide having a perovskite structure or perovskite-like structure. See U.S. Pat. No. 6,569,226 in the name of Stephen E. Dorris, Tae H. Lee, and Uthamalingam Balachandran, the entire disclosure of which is incorporated herein by reference. Membranes for use in flow reactors of the invention may be in sheet form or tubular form or honeycomb form, such as illustrated in U.S. Pat. No. 5,356,728 in the name of Uthamalingam Balachandran, Roger B. Poeppel, Mark S. Kleefisch, Thaddeus P. Kobylinski and Carl A. Udovich, the entire disclosure of which is incorporated herein by reference.

Materials known as “perovskites” are a class of materials that have an X-ray identifiable crystalline structure based upon the structure of the mineral perovskite, CaTiO₃. In its idealized form, the perovskite structure has a cubic lattice in which a unit cell contains metal ions (A) at the corners of the cell, another metal ion (B) in its center and oxygen ions at the center of each cube face. In known crystalline materials of the perovskite class, the unit cell repeats, without interruption throughout a crystal, in all three orthogonal directions of a suitably oriented rectangular coordinate system. This cubic lattice is identified as an ABO₃-type structure where A and B represent metal ions. In the idealized form of perovskite structures, generally, it is required that the sum of the valences of A ions and B ions equal 6, as in the model perovskite mineral, CaTiO₃.

Many materials having the perovskite-type structure (ABO₃-type, i.e., where β is zero) have been described in recent publications including a wide variety of multiple cation substitutions on both the A and B sites said to be stable in the perovskite structure. Likewise, a variety of more complex perovskite compounds containing a mixture of A metal ions and B metal ions (in addition to oxygen) are reported. Materials and methods useful in dense ceramic membrane preparation of the invention are described in U.S. Pat. Appl. Pub. No.: US 2005/0222479 A1, which publication is hereby incorporated herein by reference for its disclosure relating to preparation of dense ceramic membranes. Other publications relating to dense ceramic membranes include, for example P. D. Battle et al., J. Solid State Chem., 76, 334 (1988); Y. Takeda et al., Z. Anorg. Allg. Chem., 550/541, 259 (1986); Y. Teraoka et al., Chem. Lett., 19, 1743 (1985); P. D. Battle et al., J. Solid State Chem., 76, 334 (1988); M. Harder and H. H. Muller-Buschbaum, Z. Anorg. Allg. Chem., 464, 169 (1980); C. Greaves et al., Acta Cryst., B31, 641 (1975).

Any of a variety of methods may be used to make inorganic crystalline materials as described herein above. According to the present invention, useful multiphasic systems advantageously comprise two or more phases bound to one another. At least one of the bound phases demonstrates an ability to selectively convey hydrogen; another phase demonstrates an ability to selectively convey oxygen ions between different gaseous mixtures; and one or more of the phases demonstrates electronic conductivity.

Processes of the present invention allow for the presence of a catalyst to enhance conversion. Conventional steam cracking is performed without the use of catalysts, because thermodynamic equilibrium is not the object. Free radicals are produced to initiate a chain reaction. Catalysts are not used in ethane service because the equilibrium constant is too low for commercial use at temperatures below 900° C. Where temperatures exceeded, free radical reactions have taken off. However, catalysts have been used with great success in the dehydrogenation of propane and higher hydrocarbons. Propane dehydrogenation has been practiced with high selectivity, but suffers from low conversion due to the presence of hydrogen. By use of dense membranes comprising multiphasic materials that provide independent, controllable, counter-current transport of hydrogen, electrons and oxygen, processes of this invention can advantageous use of catalysts.

Dehydrogenation elements in this invention may comprises contacting, for example, in a high conversion membrane reactor an ethane containing feedstream with a catalytic amount of a dehydrogenation catalyst in a dehydrogenation zone. Contacting is conducted under reaction conditions such that a dehydrogenation product stream containing predominantly ethylene and unreacted ethane is formed. Typically, dehydrogenation catalysts comprise a mordenite zeolite and, optionally, a metal component selected from the group consisting of gallium, zinc, ruthenium, osmium, rhodium, iridium, palladium and platinum.

This invention also contemplates addition of steam with the hydrocarbon feed to inhibit free radical formation at temperatures close to temperatures required in steam pyrolysis processes. In steam pyrolysis, steam is added to the hydrocarbon in weight ratios ranging from 1:3 to 1:1 depending on the molecular weight of the hydrocarbon. Steam addition in the present invention can lower the partial pressure of the hydrocarbon, improve alkene selectivity, and decrease coke formation on the reactor surfaces.

Processes of the invention for chemical conversion of volatile organic compounds to value added products can be operated to provide absolute pressure at the outlet of the cracking or dehydrogenation zone that exceeds normal pyrolysis operating pressures. Pyrolysis reactors usually operate with an absolute pressure at the outlet of less than 45 psia. Higher pressures are not normally favored because the population of free radicals in pyrolytic processes decreases with increased pressure due to more intermolecular collisions. If the reactor pressure is too high, the process temperature has to be increased to maintain the free radical population. Another disadvantage of higher pressure and temperature is the increase of side reactions that decrease selectivity to alkenes and enhance the formation of aromatic compounds and coke. Processes of this invention are less sensitive to pressure because hydrogen is removed from the reaction zone and thereby does not increase the pressure in the reaction zone to the same extent as conventional reactors

Hydrogen fluxes of at least 1 cm³/cm²/min are desirable to so that reactor costs and the amount of material needed to fabricate a reactor does not become excessive.

Any suitable means can be used to situate membranes between reactor chambers. The art is replete with examples of reactor chambers separated by membranes. For example, see U.S. Pat. No. 5,160,713, in which Mazanec and coworkers describe disc and hollow tube membrane reactors. Mazanec and coworkers, in U.S. Pat. No. 5,306,411, describe additional cylindrical embodiments with external circuits to enhance or slow down the reaction. Balachandran and coworkers, in U.S. Pat. No. 5,356,728, disclose embodiments of cross-flow on flat plates. Dyer and coworkers, in U.S. Pat. No. 5,599,383, describe a tubular solid-state membrane module and other embodiments such as a serpentine coil. Taylor and coworkers, in U.S. Pat. No. 5,681,373, describe planar embodiments. In U.S. Pat. No. 6,293,978 B2, Kleefisch and coworkers disclose a cylindrical embodiment for oxygen ion-conducting dense ceramic membranes. In U.S. Pat. No. 6,309,612 Balachandran and coworkers disclose embodiments that operate the two chambers of the reactor at different pressures. White and coworkers, in U.S. Pat. No. 6,454,274 B2, describe a technique for holding a ceramic tube in a metal tube sheet in order to make a multi-tube reactor (module).

Membrane reactors of a present invention generally include a first chamber having at least one entrance for flow of alkanes containing no dihydrogen and at least one exit. The first chamber is separated from second chamber that has at least one entrance for flow oxygen containing gas and at least one exit, by a multiphasic solid state oxygen, hydrogen, and electron transport dense or porous membrane material. This material is formed into a disc for reactors used to demonstrate the invention, but any shape capable of creating two separate zones would be sufficient.

Control of the ratio of the hydrogen to oxygen fluxes determines how heat is transported in the membrane reactor. In one embodiment of the present invention, combustion of hydrogen occurs mainly in the first chamber. This occurs under conditions of low hydrogen flux out of the first chamber and high oxygen flux into the first chamber that has low oxygen partial pressure. In another embodiment of the present invention, combustion of hydrogen occurs mainly in the second chamber. This occurs under conditions of high hydrogen flux out of the first chamber and low oxygen flux out of the second chamber, which has high oxygen partial pressure.

Heat required to drive the endothermic dehydrogenation reactions potentially con be supplied by numerous sources, including external heating media. Another embodiment of the present invention controls the feed rate to a specific reactor so that the combustion of hydrogen provides all of the endothermic heat of reaction. For a specific alkane feed rate and conversion, a specific amount of hydrogen needs to be removed from the reactor and a specific amount of heat needs to be supplied to the first reactor chamber. The preferred process for carrying out heat-balanced simultaneous exothermic and endothermic reactions in a membrane reactor with a multiphase solid state oxygen, hydrogen, and electron transport membrane would occur if the reactor had area A=(r*ΔH_{endo}*V)/(j*ΔHₑₓₒ) square meters, wherein the ion flux through the membrane is j moles of ion per square meters per second, the reactor volume is V cubic meters, the rate of reaction is r moles of alkane per cubic meters per second, the endothermic reaction requires ΔH_{endo} watts per mole of dehydrogenatable alkane, and the exothermic reaction generates ΔHₑₓₒ watts per mole of ion. Reactors according to the present invention will meet this area requirement when the combustion of hydrogen balances all of the required endothermic heat of reaction. Numerous reactor geometries are possible according to the spirit of the present invention.

Those skilled in the art will recognize that a network of simultaneous reactions will occur in the reactor with a total heat requirement that can be supplied by the combustion of hydrogen.

### EXAMPLES OF THE INVENTION

General

The present invention is described by way of a number of relevant examples. In two-zone membrane reactors for demonstration of the invention, a hydrocarbon feed was introduced onto the upper surface of a membrane disk and air or dinitrogen was introduced onto the lower surface of the membrane disk. Principles of operation are the same for a tube or cylindrical shaped membrane reactors.

### Example 1

A multiphasic, solid state, hydrogen, electrons and oxygen transport membrane was fabricated from cerium-stabilized zirconia and palladium (CEZ/Pd) as follows:

a) 5.3 g of cerium stabilized zirconia, obtained from American Vermiculite Corporation (CEZ-10SD), was mixed with 12.06 g of palladium flake, obtained from Degussa Corporation, for 30 minutes in a mortar and pestle.

b) Approximately 5 g of the mixture was loaded into a cylindrical dye (1.25 inch diameter) and compressed to 26,000 lbs. using a Carver Laboratory Press (Model #3365).

c) The CEZ/Pd disc was sintered by heating in air to 1300° C. and holding at that temperature for 4 hours.

The sintered membrane was placed between two gold rings and heated to 900° C. at 0.5° C./minute. The sintered membrane was sealed with gold rings into the two-zone disc reactor.

Hydrogen and oxygen permeabilities were measured at 0.1-0.33 sccm/cm²/min and 0.01 sccm/cm²/min, respectively. The trans-membrane oxygen to hydrogen ratio was measured to be 0.03-0.1 and the ceramic to metal ratio was 2.45.

The reactor was heated to 800° C. under nitrogen. One side of the reactor was exposed to air and the other side exposed to ethane and steam (ethane and steam in a 1:1 weight ratio). The product from the hydrocarbon side was analyzed by gas chromatography. The carbon weight percent composition of the product is presented in Table 1.

The selectivity for ethylene was 88 percent. The ethylene production rate was 28 mL/cm²/min. This membrane was studied for 600 hours in ethane/steam service and was stable.

### Example 2

A multiphasic, solid state, hydrogen, electrons and oxygen transport membrane was fabricated from cerium gadolinium oxide and silver/palladium (CGO/(Ag/Pd)) as follows:

a) A batch of cerium gadolinium oxide powder, obtained from Rhodia, was heated in air to 1000° C. and held at that temperature for one hour. The powder was then sifted with a 60-mesh filter.

b) 1.93 g of the sifted cerium gadolinium oxide powder was mixed with 2.13 g of palladium/silver (70/30) flake, obtained from Degussa Corporation, for 30 minutes in a mortar and pestle.

c) Approximately 6 g of the mixture was loaded into a cylindrical dye (1.25 inch diameter) and compressed to 26,000 lbs. using a Carver Laboratory Press (Model #3365).

d) The CGO/(Ag/Pd) disc was sintered by heating in air to 1300° C. and holding at that temperature for 4 hours.

Hydrogen and oxygen permeabilities were measured at 12.2 sccm/cm²/min and 0.34 sccm/cm²/min, respectively. The trans-membrane oxygen to hydrogen ratio was measured to be 0.03 and the ceramic to metal ratio was 1.50 (2.14 for active metal).

The reactor was heated to 800° C. under nitrogen. One side of the reactor was exposed to air and the other side exposed to ethane and steam (ethane and steam in a 1:1 weight ratio). The product from the hydrocarbon side was analyzed by gas chromatography. The carbon weight percent composition of the product is presented in Table 1.

The selectivity for ethylene was 82 percent. The ethylene production rate was 28 mL/cm²/min. The material with the higher trans-membrane oxygen to hydrogen flux had lower conversion but higher selectivity to ethylene. The ability to control carbon monoxide, methane, acetylene, and heavier hydrocarbon production with this ratio is an economically valuable attribute.

### Example 3

In these experiments, 3-A and 3-B, the side of the membrane usually exposed to air was exposed to air and to nitrogen, respectively, to compare the role of oxygen transport.

These data indicate that, at some low level, oxygen transported into the reaction chamber suppresses methane to carbon monoxide conversion, by combusting surface carbon and keeping the reaction at the surface carbon deposition rate. In the absence of surface oxygen, the carbon monoxide formation rate is controlled by the surface carbon water gasification rate on the much larger mass of surface carbon. It is believed that at higher oxygen transport levels, the oxygen could increase carbon monoxide production through hydrocarbon combustion. Consumption of hydrogen by the oxygen advances the conversion of ethane and the dilution effect increases the selectivity to ethylene.

TABLE 1
Selectivity pattern of ethane from membrane reactors

Selectivity

| MEMBRANE | Example 1 | Example 2 |
|---|---|---|
| Conversion | 75.6% | 88.2% |
| Temperature | 878° C. | 885° C. |
| Material | CEZ/Pd | CGO/(Ag/Pd) |
| Sweep | Air | Air |
| CO | 0 | 0.5 |
| Methane | 5.2 | 8.8 |
| Ethane | | |
| Ethylene | 88.15 | 81.8 |
| Acetylene | 1.88 | 2.57 |
| Propane | 0.20 | |
| Propylene | 1.06 | 1.12 |
| Propadiene | 3.51 | 3.19 |
| Pentenes | | 2.02 |
| O2/H2 flux | 0.1 | 0.03 |
| C2 = production rate | 28 | 28 |
| (mL/cm²/min) | | |

TABLE 2
Selectivity pattern of ethane from membrane reactors

Selectivity

| EXAMPLE 3 | 3-A | 3-B |
|---|---|---|
| Conversion | 83% | 81.2% |
| Temperature | 874° C. | 873° C. |
| Material | CGO/(Ag/Pd) | CGO/(Ag/Pd) |
| Sweep | Air | N2 |
| CO | 0.5 | 2.4 |
| Methane | 5.6 | 4.9 |
| Ethane | | |
| Ethylene | 86.7 | 85.9 |
| Acetylene | 2.49 | 2.52 |
| Propane | | |
| Propylene | 0.96 | 0.76 |
| Propadiene | 2.82 | 2.65 |
| Iso-Butenes | 0.29 | |
| Pentenes | 0.64 | 0.87 |
| O2/H2 flux | 0.31 | 0 |
| C2 = production rate | 28 | 28 |
| (mL/cm²/min) | | |

### Example 4

In these experiments, 4-A and 4-B, the yields from ethane dehydrogenation were comparable, except for the slightly lower production of carbon oxides in 4-A relative to 4-B which was due to the lower trans-membrane oxygen to hydrogen flux ratio relative to experiment 4-B. The production rate of carbon oxides in both of these examples is below that measured in Example 3, Table 2 and Experiment 3-B in the absence of oxygen. The observed olefin yields in these examples of the invention significantly exceed the olefins yields obtained by state of the art pyrolysis reactors.

The metal loading was higher in the material used in Experiment 4-B was higher than that of the material used in Experiment 4-A. This made the hydrogen flux larger and the trans-membrane oxygen to hydrogen flux ratio lower. The ceramic to metal loading can be used to control the trans-membrane oxygen to hydrogen flux ratio.

TABLE 3
Selectivity pattern of ethane from membrane reactors

Selectivity

| EXAMPLE 4 | 4-A | 4-B |
|---|---|---|
| Conversion | 86.6% | 86.9% |
| Temperature | 873° C. | 884° C. |
| Material | CGO/(Ag/Pd) | CGO/(Ag/Pd) |
| CO | 0.5 | 1.2 |
| Methane | 7.3 | 6.1 |
| Ethane | | |
| Ethylene | 84.9 | 84.5 |
| Acetylene | 2.61 | 3.27 |
| Propane | | |
| Propylene | 1.1 | |
| Propadiene | 3.59 | 2.9 |
| Pentenes | | 2.02 |
| O2/H2 flux | 0.03 | 0.06 |
| C2 = production rate | 28 | 28 |
| (mL/cm²/min) | | |

### Example 5

In experiments 5-A and 5-B, the yields from propane dehydrogenation were roughly the same. Beneficially, the total olefin yields significantly exceed the olefins yields obtained from state of the art dehydrogenation and pyrolysis reactors.

TABLE 4
Selectivity pattern of propane from membrane reactors

Selectivity

| EXAMPLE 5 | 5-A | 5-B |
|---|---|---|
| Conversion | 95.0% | 94.4% |
| Temperature | 870° C. | 875° C. |
| Material | CGO/(Ag/Pd) | CGO/(Ag/Pd) |
| CO | 0.5 | 0.6 |
| Methane | 27.6 | 27 |
| Ethane | 2.3 | 2.0 |
| Ethylene | 52.83 | 51.6 |
| Acetylene | 2.26 | 2.7 |
| Propane | | |
| Propylene | 10.7 | 10.7 |
| Propadiene | 3.69 | 3.46 |
| Butenes | 0.12 | 0.6 |
| Pentenes | | 1.34 |
| O2/H2 flux | 0.03 | 0.06 |
| C2 = production rate | 27 | 27 |
| (mL/cm²/min) | | |

In experiments 5-C and 5-D, the ethane and propane feedstreams were replaced with other hydrocarbons, in particular with iso-butane and debutanized natural gasoline (DNG), a liquid cut consisting of hydrocarbons with 5 to 7 carbons and no olefins. Table 5 presents information for iso-butane fed to a membrane reactor whose perovskite phase had the composition represented by Ce_{0.8}Gd_{0.2}O_{δ}.

TABLE 5
Selectivity of iso-butane from a membrane reactor

Selectivity

| EXAMPLE 5 | 5-C | 5-D |
|---|---|---|
| Conversion | 68.3% | 80.3% |
| Temperature | 830° C. | 850° C. |
| Material | CGO/Pd | CGO/Pd |
| CO | 0.5 | 0.5 |
| Methane | 21.1 | 22.5 |
| Ethane | 1.3 | 1.6 |
| Ethylene | 10.1 | 12.9 |
| Acetylene | 0.8 | 1.2 |
| Propane | | |
| Propylene | 33.6 | 29.9 |
| Propadiene | 0.44 | 0.63 |
| 1,3-Butadiene | 2.16 | 8.09 |
| 1-Butene | 3.14 | 2.84 |
| Isobutylene | 25.7 | 18.92 |
| Pentenes | 1.16 | 0.92 |
| O2/H2 flux | 0.03 | 0.06 |
| C2 = production rate | 8 | 8 |
| (mL/cm²/min) | | |

In Example 1 the trans-membrane oxygen to hydrogen flux ratio was 0.1 with a ceramic to metal ratio of 2.45. In Examples 5-C and 5-D the trans-membrane oxygen to hydrogen flux ratio was 0.03 with a ceramic to metal ratio of 1.5. These Examples illustrate that the composition can be used to adjust the trans-membrane oxygen to hydrogen flux ratio.

That which is claimed is:

1. A process for chemical conversion of volatile organic compounds to value added products, which process comprises:
(A) Providing a solid state membrane having first surface and opposite thereto a second surface, the membrane comprising a multiphasic material that demonstrates an ability to selectively convey electrons, hydrogen and oxygen between different gaseous mixtures;
(B) Contacting the first surface of the membrane with gaseous organic stream, substantially free of dioxygen, comprising one or more volatile hydrocarbon;
(C) Contacting the second surface of the membrane with a gaseous non-organic composition, substantially free of dihydrogen and organic compounds, comprising at least 5 percent by volume of dioxygen;
(D) Converting, at elevated temperatures, one or more volatile compound in the organic stream to products of conversion comprising corresponding value added organic products, carbonaceous co-products, and hydrogen;
(E) Permitting at least a portion of the hydrogen co-product to be selectively conveyed through the membrane, from the first surface to the second surface, and oxidizing the conveyed hydrogen; and
(F) Permitting a predetermined amount of oxygen, in the form of oxygen ions, to be selectively conveyed through the membrane from the second surface to the first surface.

2. The process according to claim 1 wherein at least a portion of the carbonaceous co-products are oxidized, on or near the first surface, by oxygen selectively conveyed through the membrane from the second surface.

3. The process according to claim 1 wherein the conversions are carried out in the presence of a hydrocarbon conversion catalyst at elevated temperatures in a range from about 400° C. to about 900° C.

4. The process of claim 1 wherein a flux of the co-product hydrogen conveyed through the membrane, from the first surface to the second surface of membrane is at least 1 cm³/min. at standard conditions per cm² of membrane area.

5. The process of claim 4 wherein a flux of oxygen conveyed through the membrane from the second surface to the first surface is no more than the counter-current flux of hydrogen.

6. A hydrocarbon dehydrogenation process for the production of olefins, the process comprising:
(A) Providing a gaseous feedstream consisting predominantly of volatile alkane compounds substantially free of dihydrogen and/or dioxygen;
(B) Providing a solid state membrane having first surface and opposite thereto a second surface, the membrane comprising a non-homogenous, multiphasic solid containing a first phase comprising a metal, alloy or mixed-metal oxide, and a second phase comprising a mixed metal oxide ceramic, wherein the first and second phases are bound to one another and distributed, in a physically distinguishable form, throughout the continuous, fine-grained, second phase, wherein at least one of the bound phases demonstrates an ability to selectively convey hydrogen, another phase demonstrates an ability to selectively convey oxygen ions between different gaseous mixtures, and one or more of the phases demonstrates electronic conductivity;
(C) Converting, under conditions of reaction for hydrocarbon dehydrogenation at elevated temperatures in a reaction mixture, one or more alkane hydrocarbon in the gaseous feedstream to products of dehydrogenation comprising corresponding value added alkene hydrocarbons, carbonaceous co-products, and hydrogen;
(D) Contacting the first surface of the membrane with gaseous reaction mixture;
(E) Contacting the second surface of the membrane with a gaseous non-organic composition, substantially free of dihydrogen and organic compounds, comprising from about 5 upward to about 30 percent by volume of dioxygen;
(F) Permitting a predetermined amount of oxygen, in the form of oxygen ions, to be selectively conveyed through the membrane from the second surface to the first surface; and
(G) Permitting at least another portion of the co-product hydrogen to be selectively conveyed through the membrane, from the first surface to the second surface, and oxidizing the conveyed hydrogen.

7. The process according to claim 6 wherein at least a portion of the carbonaceous co-products are oxidized, on or near the first surface, by oxygen selectively conveyed through the membrane from the second surface.

8. The process according to claim 7 wherein the gaseous feedstream comprises volatile alkane compounds having from about 1 to about 8 carbon atoms, and the conversions are carried out at elevated temperatures in a range from about 400° C. to about 900° C. and pressures in a range upward from about 15 psia to about 500 psia.

9. The process of claim 7 wherein the product alkene compounds comprise at least one member of the group consisting of ethylene, propylene, and isomers of butene.

10. The process of claim 6 wherein the gaseous non-organic composition further comprise at least one member of the group consisting of dinitrogen and carbon dioxide.

12. A dehydrogenation process for the production of olefin hydrocarbons from a gaseous feedstream consisting predominantly of volatile alkane compounds, the process comprising:
(A) Providing apparatus comprising a plurality of membrane modules each including first and second zones separated by a membrane comprising a multiphasic material comprising a non-homogenous, multiphasic solid containing a first phase comprising a metal, alloy or mixed-metal oxide, and a second phase comprising a mixed metal oxide ceramic, wherein the first and second phases are bound to one another and distributed, in a physically distinguishable form, throughout the continuous, fine-grained, second phase, wherein at least one of the bound phases demonstrates an ability to selectively convey hydrogen, another phase demonstrates an ability to selectively convey oxygen ions between different gaseous mixtures, and one or more of the phases demonstrates electronic conductivity, each first zone having at least one inlet and outlet for flow of fluid in contact with the membrane, and contiguous with the opposite side thereof a second zone having at least one outlet for flow of another fluid;
(B) Introducing a feedstream comprising volatile alkane compounds, substantially free of dihydrogen and/or dioxygen into the first zone of one or more of the modules;
(C) Introducing a gaseous non-organic stream substantially free of dihydrogen and organic compounds, and comprising from about 5 upward to about 30 percent by volume of dioxygen into the second zone of one or more of the modules;
(D) Converting, under conditions of dehydrogenation at elevated temperatures in the first zone of one or more of the modules, one or more alkane hydrocarbon in the feedstream to corresponding value added alkene hydrocarbons, carbonaceous co-products, and hydrogen;
(E) Permitting at least a portion of the hydrogen co-product to be selectively conveyed through the membranes, from the first zones into the second zones; and
(F) Permitting a predetermined amount of oxygen, in the form of oxygen ions, to be selectively conveyed through the membrane from the second zones into the first zones.

13. The process according to claim 12 wherein conversions of alkane hydrocarbon in the feedstream carried out in the first zones is at least 75 molar percent.

14. The process according to claim 12 wherein at least a portion of the carbonaceous co-products are oxidized, on or near the first surface, by oxygen selectively conveyed through the membrane from the second surface.

15. The process of claim 12 wherein the first phase comprises a metal selected from the group consisting of silver, palladium, platinum, gold, rhodium, titanium, nickel, ruthenium, tungsten, and tantalum.

16. The process of claim 12 wherein the first phase comprises a ceramic selected from the group consisting of a praseodymium-indium oxide mixture, niobium-titanium oxide mixture, titanium oxide, nickel oxide, tungsten oxide, tantalum oxide, ceria, zirconia, magnesia, or a mixture thereof.

17. The process of claim 12 wherein the second phase comprises a mixed conducting oxide composition represented by
(A_{1-y}A′_{y})(B₁₋ₓB′_{z}B″_{x-z})O_{δ}
where A is a lanthanide element, yttrium (Y), or mixture thereof, A′ is one or more alkaline earth metal; B is iron (Fe); B′ is chromium (Cr), titanium (Ti), or mixture thereof and B″ is manganese (Mn), cobalt (Co), vanadium (V), nickel (Ni), copper (Cu) or mixture thereof; and x and y are each independently selected numbers from zero to about one, and z is a number zero to x; and δ is a number determined from stoichiometry that renders the compound charge neutral.

18. The process of claim 12 wherein the second phase comprises a mixed cerium oxide composition represented by
M′CeO_{δ}
where M′ is selected from the group consisting of yttrium (Y) and elements having atomic numbers from 58 to 71 inclusive, and δ is a positive number determined from stoichiometry.

19. The process of claim 12 wherein the second phase comprises a mixed zirconium oxide composition represented by
M″ZrO_{δ}
where M″ is selected from the group consisting of calcium (Ca), yttrium (Y) and elements having atomic numbers from 58 to 71 inclusive, and δ is a positive number determined from stoichiometry.

20. The process of claim 12 wherein a flux of the co-product hydrogen conveyed through the membrane, from the first zones to the second zones of the modules is at least 1 cm3/cm2/min, and a flux of oxygen conveyed through the membrane from the second zones to the first zones is no more than about one-tenth of the counter-current hydrogen flux.

21. The process of claim 12 wherein at least a plurality of the membrane modules further comprise structures that during operation facilitate an external path for flow of electrons.
